(19) United States
(12) Patent Application Publication
(10) Pub. No.: US 2004/0151785 A1
(43) Pub. Date: Aug. 5, 2004

(54) Method for treating insulin resistance through hepatic nitric oxide

(75) Inventor: Wilfred Wayne Lautt, Winnipeg (CA)

Correspondence Address: MERCHANT & GOULD PC, P.O. BOX 2903, MINNEAPOLIS, MN, 55402-0903, US

(73) Assignee: DiaMedica Inc., Winnipeg (CA)

(21) Appl. No.: 10/761,596

(22) Filed: Jan. 21, 2004

Related U.S. Application Data
(63) Continuation of application No. 09/806,989, filed on May 29, 2001.
Application No. 09/806,989 is a 371 of international application No. PCT/US99/23098, filed on Oct. 5, 1999.
(60) Provisional application No. 60/103,170, filed on Oct. 6, 1998.

(51) Int. Cl. A61K 33/00

(52) U.S. Cl. 424/718

(57) ABSTRACT

There is provided a method of increasing insulin sensitivity by administering an effective amount of a compound which stimulates nitric oxide production in the liver. Also provided is a pharmaceutical composition having an effective amount of a compound which stimulates nitric oxide production in the liver and a pharmaceutically acceptable carrier.

### FIELD OF THE INVENTION

The present invention relates to a compound and method for the treatment of insulin resistance.

### BACKGROUND OF THE INVENTION

Patients with non-insulin dependent diabetes mellitus (NIDDM) show insulin resistance, impaired glucose tolerance, and parasympathetic neuropathies. Several other disease states are also associated with the co-existence of parasympathetic neuropathies and insulin resistance. These conditions include patients with chronic essential hypertension, obesity, patients with liver disease, and patients with transplanted livers.

Chap et al., (17) demonstrated that the absorption of orally administered glucose in conscious dogs was suppressed and delayed by administration of atropine. The mechanism of this response was demonstrated using an isolated, jointly perfused small bowel and liver preparation in rats (19). Administration of insulin into the portal blood supply led to a parasympathetic nerve-mediated increase in absorption of glucose from the lumen of the intestine. The effect could be blocked by atropine and mimicked by carbachol. The afferent limb of the reflex is activated by insulin with receptors located in the portal vein or liver and the efferent limb represents muscarinic nerves supplying the intestine.

The neural pathway connecting the sensory and effector branches of the reflex is not known but, in this unique preparation, could only occur through two sources. One route is from the liver along the portal vein through the posterior hepatic plexus to the intestine. The other involves transmission through the celiac ganglion which remained intact in this preparation. Regardless of the course, this is an example of a splanchnic reflex that does not pass through the central nervous system. This mechanism likely serves the function of assuring that maximum glucose absorption only occurs at a time when the organs sensitive to insulin-induced uptake have also been stimulated.

In 1993, it was (10) first noted that the hypoglycemic response to a bolus administration (5 minute infusion) of insulin (100 mU/kg i.v.) was reduced by 37% by hepatic denervation in fasted cats. These fasted cats developed insulin resistance immediately following acute denervation of the liver. The degree of reduction of response to insulin was maximal after anterior plexus denervation and did not increase further with addition of denervation of the posterior nerve plexus or bilateral vagotomy thus demonstrating that all of the nerves of relevance were in the anterior plexus.

To avoid the complexity of the reaction to hypoglycemia, a new rapid insulin sensitivity test (RIST) was developed (20) wherein a euglycemic clamp was used following the administration of insulin and the response was quantitated as the amount of glucose required to be infused over the test period in order to hold arterial blood glucose levels constant. The RIST methodology has been published in detail (20) and has been demonstrated in both cats and rats. It is highly reproducible with up to five consecutive responses being obtainable in cats and four in rats with blood glucose levels returning to control levels between each test. Insulin, glucagon, and catecholamine levels remain unchanged between tests.

Cats showed a dose-related development of insulin resistance using atropine (11) that was of a similar magnitude to that produced by surgical denervation. The dose of atropine required to produce a full insulin resistance is 3 mg/kg (4 μmol/kg) administered into the portal vein. A similar degree of insulin resistance was achieved with 10⁻⁷ mmol/kg of the M₁ muscarinic selective antagonist, parenzepine, and with 10⁻⁸ μmol/kg of the M₂ selective antagonist, methoctramine. Although not conclusive, the data suggest that the response may be mediated by the M₁ muscarinic receptor subtype (21).

Although the liver was clearly the organ that produced the insulin resistance, it was not clear that the liver was the resistant organ. In order to determine the site of insulin resistance, a further series was done in cats that measured arterial-venous glucose responses across the hindlimbs, extrahepatic splanchnic organs, and liver (22). The intestine was unresponsive to the bolus insulin administration both before and after atropine or anterior plexus denervation or the combination of both. The hepatic response was also not notably altered whereas the glucose uptake across the hindlimbs, primarily representing skeletal muscle uptake, was decreased following atropine or hepatic parasympathetic denervation. These results indicated that interference with hepatic parasympathetic nerves led to insulin resistance in skeletal muscle.

It was further demonstrated that the same degree of resistance could be produced by pharmacological blockade of parasympathetic nerve function using the muscarinic receptor antagonist, atropine (Xie et al., 1994; Xie and Lautt, 1995; Xie and Lautt, 1996a, 1996b). Also, the ability to reverse insulin resistance produced by interruption of hepatic parasympathetic nerve function and to reverse insulin resistance in a model of liver disease led to the issue of U.S. Pat. No. 5,561,165 on Oct. 1, 1996. Accordingly, it was determined that, following a meal, insulin is released from the pancreas. The presence of insulin in the blood elicits a hepatic parasympathetic reflex that results in the release of acetylcholine in the liver that results in the generation and release of nitric oxide which, in turn, results in the release of an active hormone (hepatic insulin sensitizing substance (HISS)) into the blood. HISS controls the sensitivity of skeletal muscle to insulin so that in its presence, the muscle (and probably other tissues) is extremely sensitive to the effects of insulin and results in a rapid uptake and storage of glucose.

In the absence of HISS, the large muscle mass is highly resistant to insulin and the glucose storage in skeletal muscle is severely reduced. Interruption of any part of the parasympathetic-mediated release of HISS results in insulin resistance. This parasympathetic reflex regulation of HISS release is the fundamental mechanism by which the body regulates the responsiveness to insulin and this mechanism is adjusted according to the prandial state, that is, according to how recently there has been a consumption of nutrients.

In a fasted condition, HISS release in response to insulin is minimal or absent so that if insulin is released in this situation, there is a minimal metabolic effect. Following a meal, the parasympathetic reflex mechanism is amplified so that HISS release occurs and results in the majority of the ingested glucose being stored in skeletal muscle.

The consequence of lack of HISS release is the absence of HISS results in severe insulin resistance. In this situation, the pancreas is required to secrete substantially larger amounts of insulin in order that the glucose in the blood is disposed of to prevent hyperglycemia from occurring. If this condition persists, insulin resistance will progress to a state of type 2 diabetes (non-insulin dependent diabetes mellitus) and eventually will lead to a complete exhaustion of the pancreas thus requiring the patient to resort to injections of insulin. Thus, any condition in which the hepatic parasympathetic reflex is dysfunctional will result in insulin resistance.

There is evidence that the insulin resistance that is seen in a variety of conditions (non-insulin dependent diabetes, essentially hypertension, obesity, chronic liver disease, fetal alcohol effects) results from a hepatic parasympathetic dysfunction. Lack of HISS would also be anticipated to result in obesity at the early stage of the resultant metabolic disturbance (the obese often become diabetic).

Normally after a meal, the liver takes up a small proportion of glucose and releases HISS to stimulate skeletal muscle to take up the majority of the glucose load. In the absence of HISS, the skeletal muscle is unable to take up the majority of glucose thus leaving the liver to compensate. The hepatic glycogen storage capacity is insufficient to handle all of the glucose, with the excess being converted to lipids which are then incorporated into lipoproteins and transported to adipose tissue for storage as fat. Provision of HISS to these individuals would restore the nutrition partitioning so that the nutrients are stored primarily as glycogen in the skeletal muscle rather than as fat in the adipose tissue.

A major finding of direct relevance for designing therapeutic approaches was that acetylcholine infused directly into the portal vein (2.5 μg/kg/min) resulted in a complete reversal of the insulin resistance induced by surgical denervation. Administration of the same dose of acetylcholine intravenously produced no reversal. Intraportal administration directly targets the liver whereas intravenous infusion bypasses the liver and is not organ selective. This demonstration is extremely important in that the data indicate that the signal from th liver to skeletal muscle is blood-borne. This blood-borne signal is referred to as the hepatic insulin sensitizing substance (HISS). However, there has been no evidence of compounds which can be used to control or alter this pathway.

It would, therefore, be useful to determine methods and compounds for reversing insulin resistance by affecting the insulin resistance pathway.

### SUMMARY OF THE INVENTION

According to the present invention, there is provided a method of increasing insulin sensitivity by administering an effective amount of a compound which stimulates nitric oxide production in the liver. Also provided is a pharmaceutical composition having an effective amount of a compound which stimulates nitric oxide production in the liver and a pharmaceutically acceptable carrier.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a bar graph showing the rapid insulin sensitivity test (RIST) index before and after intravenous L-NAME administration and two hours after administration;

FIGS. 2A and 2B are graphs showing (A) the control RIST index versus the change from control after L-NAME administration; and (B) the control RIST index versus the change from control after parasympathectomy and intraported atropine administration;

FIG. 3 is a bar graph showing the RIST index in a control, after intraportal or intravenous L-NAME administration, and after intraportal atropine administration;

FIG. 4 is a bar graph showing the RIST index in control, after parasympathetic denervation, and after intraportal L-NMMA administration;

FIG. 5 is a bar graph showing the RIST index in a control, and after intravenous L-NAME and intraportal L-arginine administration;

FIG. 6 is a bar graph showing the RIST index in a control, after intraportal L-NMMA administration and two hours post L-NMMA administration;

FIG. 7 is a bar graph showing the RIST index in a control and after intraportal L-NMMA and intraportal SIN-1 administration;

FIG. 8 is a bar graph showing the RIST index in a control and after intraportal L-NMMA and intraportal SIN-1 administration; and

FIG. 9 is a bar graph showing the RIST index in a control, after parasympathetic denervation, and after intraportal SIN-1 administration.

### DETAILED DESCRIPTION

Generally, the present invention provides a compound and method of increasing insulin sensitivity by administering an effective amount of a compound which stimulates nitric oxide production in the liver. More specifically, the compound can be administered as a nitric oxide donor or as a stimulus that generates nitric oxide within the liver. Therefore, this compound and method can be useful in treating obesity, insulin resistance, and other diseases associated with insulin resistance.

The compounds of the present invention can be considered, generally, as members of the groups of nitric oxide agonists and NO donors. Examples of such compounds include, but are not limited to: 3-morpholinosyndnonimine (SIN-1), sodium nitrite, nitroprusside, S-nitroso-N-acetyl-D, L-penicillamine (SNAP).

It was recently demonstrated that there is a powerful hepatic parasympathetic reflex in response to insulin. Insulin results in a hepatic parasympathetic activation of cholinergic muscarinic receptors which lead to release of a hepatic insulin sensitizing substance (HISS) that enters the bloodstream and regulates insulin sensitivity in skeletal muscle. Virtually all of the variability in insulin sensitivity in fed rats is demonstrated to be due to variability in the hepatic parasympathetic-dependent insulin response. Insulin resistance is produced by surgical or pharmacological blockade of the hepatic parasympathetic nerves and is easily demonstrated using a new insulin sensitivity test. The insulin resistance so produced does not affect the splanchnic organs but appears to be restricted to skeletal muscle and, therefore, strongly resembles the sort of insulin resistance seen in non-insulin-dependent diabetes mellitus and in patients with chronic liver disease.

Insulin resistance produced by surgical denervation of the liver or the chronic bile duct ligation model of liver disease can be restored completely to normal levels by intraportal but not intravenous administration of acetylcholine. It is shown that many forms of insulin resistance in different disease states are secondary to hepatic parasympathetic neuropathy. This pathway shows an unexpected but major role for hepatic parasympathetic nerves in physiological and pathological regulation of glucose metabolism.

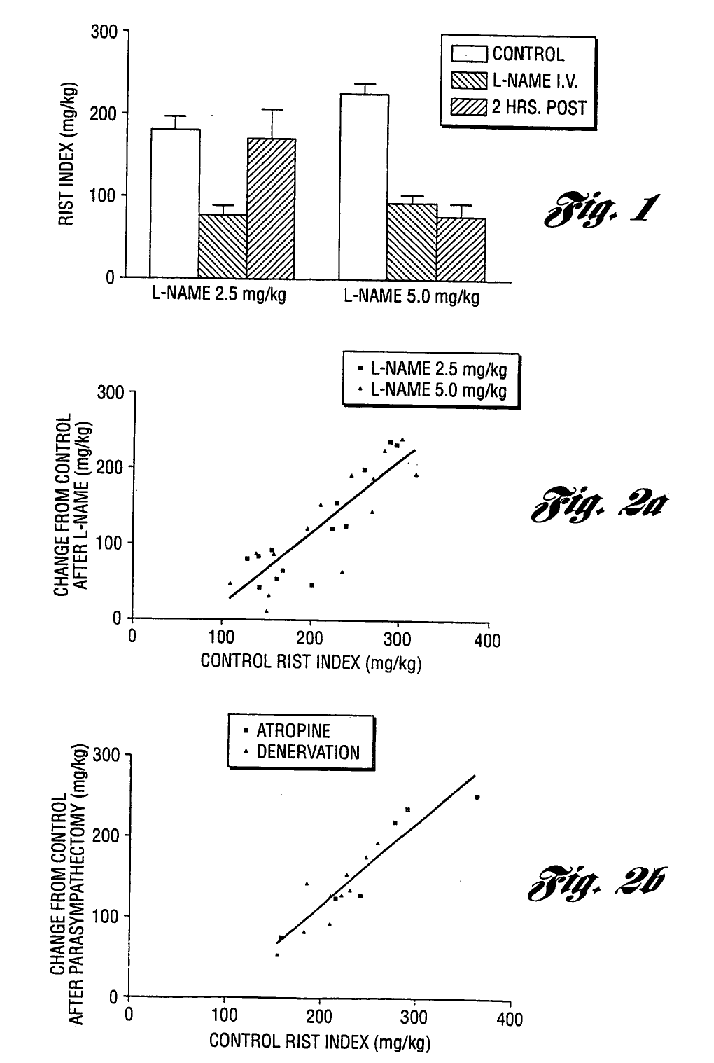

A recent series of studies reported that insulin initiates a parasympathetic reflex which results in the release of acetylcholine (Ach) in the liver (26-29). Ach acts on muscarinic receptors and causes the release of a hepatic insulin sensitizing substance (HISS). HISS enters the blood and sensitizes the skeletal muscle response to insulin. Since many cholinergic effects are mediated through nitric oxide (NO), the hypothesis that this parasympathetic effect is also mediated through NO (FIG. 1) was tested.

To quantify insulin sensitivity in rats a modified euglycemic clamp method for conducting a rapid insulin sensitivity test (RIST) (29) was used. Interruption of the hepatic reflex response to insulin by surgical denervation of the liver or atropine results in instant and reversible (26-28) insulin resistance in skeletal muscle (27). To evaluate the involvement of NO, two nitric oxide synthase (NOS) antagonists were used, N-nitro-L-arginine methyl ester (L-NAME) and N-monomethyl-L-arginine (L-NMMA). The insulin resistance produced by intravenous verses intraportal NOS antagonism was also compared to determine if the liver was the site of NO action. 3-morpholinosyndnonimine (SIN-1), a NO donor, was administered intravenously or intraportally to reverse the insulin resistance produced by L-NMMA.

The results are consistent with the hypothesis that inhibition of NOS in the liver interrupts the parasympathetic reflex, resulting in insulin resistance and that NO delivered to the liver can restore insulin sensitivity to normal levels when insulin resistance is produced by blockade of NO production in the liver or surgical destruction of hepatic nerves.

The normal response to insulin is a parasympathetic reflex release of acetylcholine leading to nitric oxide generation and production of HISS. Provision of nitric oxide to the liver can result in reversal of parasympathetic neuropathy-induced insulin resistance regardless of the cause of parasympathetic neuropathy. This includes situations where no true neuropathy exists but where the primary dysfunction is with the ability to produce nitric oxide. Nitric oxide can be administered to the liver by provision of nitric oxide donors or nitric oxide agonists or compounds that generate nitric oxide within the liver when administered orally, intravenously, intramuscularly, subcutaneously, or by delivery through a pump system directly into the portal vein. Ideally such a compound would be administered prior to a meal in order to restore normal hepatic parasympathetic responses to insulin and thereby restore insulin sensitivity. The present invention therefore provides a pharmaceutical composition containing an effective amount of a compound which stimulates nitric oxide production in the liver and a pharmaceutically acceptable carrier.

The above discussion provides a factual basis for the use of compounds and methods for stimulating nitric oxide production in the liver for treating insulin resistance. The methods used with the utility of the present application can be shown by the following non-limiting examples and accompanying figures.

### GENERAL METHODS

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

Delivery of Therapeutics (Compound):

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses may be single doses or multiple doses over a period of several days, but single doses are preferred.

The doses may be single doses or multiple doses over a period of several days. Additionally, dosing can be single doses or multiple doses prior to each meal for the duration of the disease. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated.

### EXAMPLE 1

### Methods and Materials

Male Sprague-Dawley rates were fed ad-lib with standard laboratory rat chow. The rats were anesthetized with an intraperitoneal injection of phenobarbital sodium (65 mg/kg). Anesthesia was maintained throughout the experiment by continuous infusion of pentobarbital solution (1.0 ml/100 g of body weight/hr, 1.0 mg/ml) through a cannula in the venous side of the arterial-venous loop (described below). The temperature was maintained at 37.5±0.5° C. by means of a temperature controlled surgical table and a heat lamp over the table. The body temperature was monitored with a rectal probe thermometer (HI8857, Hanna Instruments). The rats were heparinized with 100 IU/kg heparin.

Surgical preparation. The left jugular vein was cannulated for glucose infusion. Spontaneous respiration was allowed through a tracheal tube. The blood samples (25 μl) were obtained through a right femoral arterial-venous loop (30). The right femoral artery was cannulated with the arterial side of the loop. The right femoral vein was cannulated with the venous side of the arterial-venous loop. Arterial blood pressure was monitored via the arterial-venous loop by clamping the silicon sleeve on the venous side of the loop. One of the advantages of using this loop is that blood samples can be taken directly from a moving stream of blood with no need to wash or flush sampling catheters. The arterial blood continuously flows through the loop into the venous side. Intravenous infusions, except glucose, were given through the venous side of the loop. After laparotomy, the portal vein was cannulated with a 24G (OPTIVA™, Johnson & Johnson Medical Inc.) intravenous catheter for intraportal drug administration.

The rats were allowed to stabilize from the surgical interventions for at least 30 minutes before any procedures were carried out. Arterial blood samples were taken every five minutes, and glucose concentrations were immediately analyzed by the oxidase method with a glucose analyzer (model 27, Yellow Springs Instrumentals) until three successive stable glucose concentrations were obtained. The mean of these three concentrations is referred to as the basal glucose level.

Rapid Insulin Sensitivity Test (RIST). After the basal glucose level was determined, insulin (50 mU/kg in 0.5 ml saline) was intravenously infused over five minutes. Euglycemia was maintained by a variable glucose infusion. The glucose solution was prepared in saline (100 mg/ml) and infused by a variable infusion pump (Harvard Apparatus). To avoid hypoglycemia, the glucose infusion (5 mg/kg/min) was started one minute after insulin infusion. On the basis of the arterial glucose concentrations measured at two minute intervals, the infusion rate of the glucose pump was adjusted whenever required to clamp the arterial glucose levels as close to the basal value as possible. The amount of glucose infused over 30 minutes following insulin administration represents the magnitude of insulin sensitivity and is referred to as the RIST index. This method has previously been described (30) and a standard operating procedure is given (11).

Rapid Insulin Sensitivity Test time controls. The control RIST was repeated three times in the same animal (n=5). The rats were allowed to stabilize between each RIST.

Rapid Insulin Sensitivity Test in control and after L-NAME at doses 2.5 mg/kg and 5.0 mg/kg intravenously. After the control RIST, L-NAME, at dose 2.5 mg/kg (n=12) or 5.0 mg/kg (n=17), was infused intravenously over five minutes. A stable basal arterial glucose concentration was determined and a RIST was performed as described above. After 30 minutes of restabilization, basal arterial glucose concentrations were determined and a second post L-NAME RIST was repeated to measure the duration of action of each dose.

Rapid Insulin Sensitivity Test in control, after L-NAME intravenously or intraportally and after Atropine. The RIST index was determined before and after L-NAME (1.0 mg/kg) was infused either intravenously (n=5) or intraportally (n=5) over five minutes. Atropine (3.0 mg/kg) was infused intraportally over five minutes and the RIST was repeated.

Rapid Insulin Sensitivity Test in control, after surgical denervation and after L-NMMA (n=3). After the control RIST, the nerve bundles around the common hepatic artery were cut and the animal was allowed to stabilize and the RIST was repeated. L-NMMA (0.73 mg/kg) was intravenously infused and the RIST was performed.

Rapid Insulin Sensitivity Test in control, after L-NAME and after L-arginine(n=6). After a control RIST, L-NAME (5 mg/kg) was infused intravenously over five minutes. After the second RIST, L-arginine (50 mg/kg) was infused intraportally and the RIST was repeated.

Rapid Insulin Sensitivity Test in control and after L-arginine (n=4). After a control RIST, L-arginine (50 mg/kg) was infused intraportally and insulin sensitivity was measured by the RIST.

Rapid Insulin Sensitivity Test in control and after L-NMMA (n=3). After the control RIST, L-NMMA (0.73 mg/kg) was infused intraportally over five minutes. After the RIST, the animal was allowed to restabilize for 30 minutes. Basal arterial glucose concentrations were determined and a second post L-NMMA RIST was repeated to measure the duration of the action of the dose.

Rapid Insulin Sensitivity Test in control, after L-NMMA and after SIN-1 intraportally or intravenously. After the control RIST, L-NMMA (0.73 mg/kg) was infused intraportally over five minutes. After the RIST, SIN-1 (5.0 mg/kg) was infused either intraportally (n=5) or intravenously (n=4) over one minute. Insulin sensitivity was measured by the RIST.

Rapid Insulin Sensitivity Test in control, after L-NMMA and after intraportal SIN-1 (n=5). After the control RIST, L-NMMA (0.73 mg/kg) was intraportally infused over five minutes. After the RIST, SIN-1 (10.0 mg/kg) was infused intraportally over two minutes and the RIST was repeated.

Rapid Insulin Sensitivity Test in control, after surgical denervation and after intraportal SIN-1 (n=6). After the control RIST, the nerve bundles around the common hepatic artery were cut and the animal was allowed to stabilize. After the RIST, SIN-1 (10.0 mg/kg) was infused intraportally over two minutes and the RIST was repeated.

Drugs. L-NAME, L-NMMA, L-arginine and atropine were purchased from Sigma Chemical (St. Louis, Mo.). SIN-1 was purchased from Alexis Corporation (San Diego, Calif.). The human insulin was obtained from Eli Lilly & Company (Indianapolis, Ind.). All the chemicals were dissolved in saline.

Data analysis. Data were analyzed using repeated-measures analysis of variance followed by Tukey-Kramer multiple comparison test in each group or, when applicable, paired and unpaired Student's t tests. The analyzed data were expressed as means±SE throughout. Some results were analyzed using linear regression analysis. Differences were accepted as statistically significant at p<0.05. Animals were treated according to the guidelines of the Canadian Council on Animal Care.

### RESULTS

The index used to express insulin sensitivity is the total amount of glucose (mg/kg) infused over 30 minutes after insulin (50 mU/kg) administration in order to maintain euglycemia at the baseline level and is referred to as the RIST index.

RIST in time controls. Three consecutive control RISTs were performed in the same animal. The RIST indexes were 207.0±17.1 mg/kg, 202.4±25.7 mg/kg and 200.5±35.0 mg/kg, respectively. There was no significant difference in glucose infusion between each RIST during the experiment. The mean coefficient of variance (standard deviation/mean RIST index for each rat) between the tests was 8.8±1.5%. The basal glucose levels before each RIST (106.1±8.0 mg/dl, 99.4±10.8 mg/dl, 106.1±11.3 mg/dl, respectively) were not significantly different. The blood pressure was stable (110±6.9 mmHg, 111.7±9.0 mmHg, 107.5±9.8 mmHg, respectively) throughout each test. Thus, all three RISTs were similar.

RIST after intravenous L-NAME infusion. The control RIST index was 178.5±16.5 mg/kg. L-NAME at dose 2.5 mg/kg (n=12) significantly reduced the RIST index to 78.1±9.8 mg/kg and caused a 56.2±6.3% inhibition of the control response. However after two hours when the RIST was repeated again, the amount of glucose required to maintain the euglycemia was 168.4±38.7 mg/kg which was not significantly different from the control RIST (FIG. 1). The blood pressure increased after L-NAME infusion from 107.6±4.7 mmHg to 133.4±5.3 mmHg but after two hours it decreased to 110.4±10.7 mmHg. The basal glucose was similar before each RIST (111.8±4.2 mg/ml, 90.4±5.0 mg/ml, 110.3±3.0 mg/ml, respectively).

In another set of animals (n=17), L-NAME at dose 5.0 mg/kg significantly reduced the control RIST index (226.9±15.3 mg/kg) to 93.7±8.7 mg/kg and caused a 55.3±5.3% inhibition of the control response. Two hours after administration, the RIST index was 75.8±16.0 mg/kg with 66.5±7.5% inhibition of the control response (FIG. 1). After L-NAME infusion, the blood pressure increased from 107.6±4.3 mmHg to 123.5±6.0 mmHg and stayed at the same level, 120.0±7.5 mmHg, after two hours. The basal glucose was similar before each RIST (117.9±3.3 mg/ml, 107.4±3.4 mg/ml, 115.6±5.3 mg/ml, respectively). Thus both 2.5 mg/kg and 5.0 mg/kg L-NAME produce similar insulin resistance but the duration of action is less than two hours with the low dose but was maintained for at least two hours for the high dose.

The change from control after L-NAME, 2.5 mg/kg (n=12) and 5.0 mg/kg (n=17), was plotted against the control RIST index (mg/kg) (FIG. 2a). The regression line has an x-intercept of 79.5 and a slope of 0.94±0.11. This relationship is interpreted to quantitate the HISS-dependent and HISS-independent component of insulin action. Rats showing the greatest response to insulin show the greatest HISS-dependent component of insulin action.

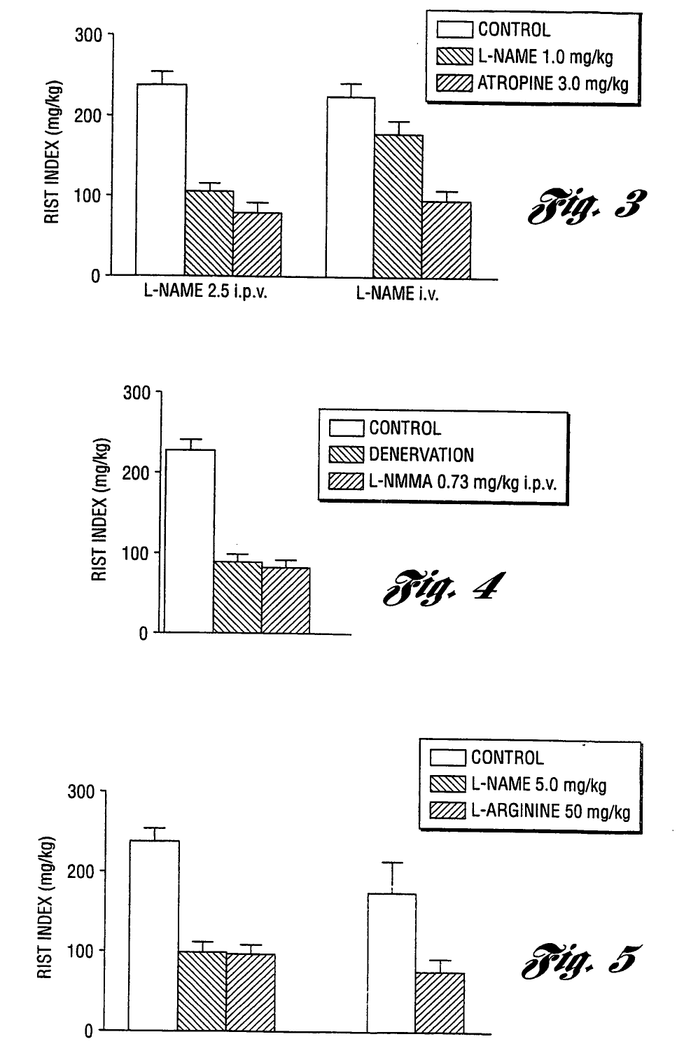

RIST after intravenous verses intraportal L-NAME. The control RIST index (n=5), of 224.1±23.5 mg/kg was not significantly reduced (177.9±21.2 mg/kg) after intravenous infusion of L-NAME (1.0 mg/kg). However administration of atropine, a non-selective muscarinic antagonist, intraportally markedly reduced the RIST index to 95.3±14.6 mg/kg and caused a56.0±8.7% inhibition of the control RIST (FIG. 3). The blood pressure was constant throughout the experiment (96.0±4.5 mmHg in control, 100.0±11.5 mmHg after L-NAME and 93.0±8.6 mmHg after atropine). In the second set of animals (n=5), the control RIST index (238.8±16.4 mg/kg) was significantly reduced by intraportal L-NAME (1.0 mg/kg) (105.8±10.8 mg/kg), causing a 54.9±5.2% inhibition of the control response. However, administration of intraportal atropine caused a further significant reduction in RIST index (78.5±14.2 mg/kg) (FIG. 3). The blood pressure increased from 99.0±1.1 mmHg to 114.0±4.5 mmHg after L-NAME but it decreased to 104±8.0 mmHg after atropine consistent with data from the 2.5 mg/kg dose showing effects wearing off by the time of the second (atropine) test. Thus, intraportal but not intravenous L-NAME at the dose of 1.0 mg/kg caused significant insulin resistance.

RIST after denervation and L-NMMA (n=3). Surgical denervation of the hepatic anterior plexus significantly reduced the RIST index from 228.3±13.8 mg/kg to 86.0±7.4 mg/kg and produced 62.0±4.8% inhibition (FIG. 4). Infusion of intraportal L-NMMA (0.73 mg/kg) did not cause a further significant reduction in RIST index (80.8±10.5 mg/kg).

The change from control RIST index after intraportal atropine (n=6) or hepatic denervation (n=10) plotted against control RIST index (mg/kg) (FIG. 2b) shows a x-intercept of 88.0 and a slope of 1.0±0.1. Insulin's action has a parasympathetic-dependent and a parasympathetic-independent component and the higher the RIST index is, the more the response is inhibited by atropine or hepatic parasympathetic denervation.

RIST after L-NAME and L-arginine (n=6). After L-NAME (5.0 mg/kg, intravenous) the RIST index was significantly reduced from 237.0±26.1 mg/kg to 99.0±12.2 mg/kg and a 55.4±8.8% inhibition of control RIST was produced. L-arginine (50 mg/kg, intraportal) administration did not reverse the inhibition by L-NAME (53.8±7.1%) (FIG. 5).

RIST after L-arginine. Following the control RIST, administration of intravenous L-arginine (50 mg/kg, n=5) significantly inhibited the control response by 48.8±8.2% (FIG. 5).

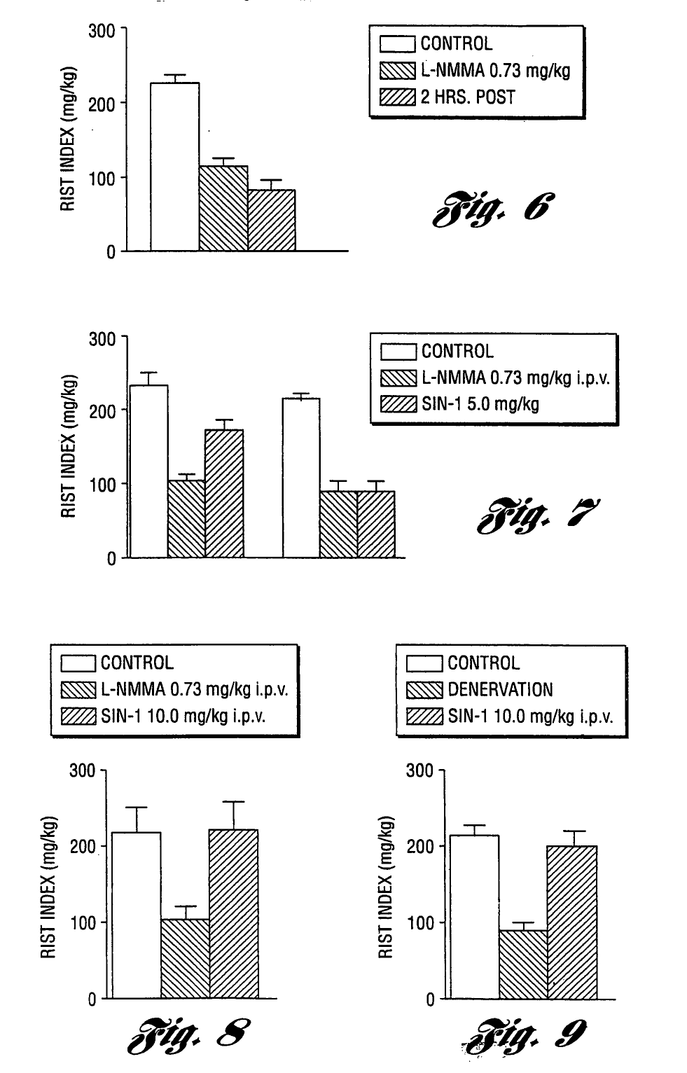

RIST after L-NMMA(n=3). Administration of intraportal L-NMMA (0.73 mg/kg) significantly reduced the RIST index from 236.8±37.6 mg/kg to 123.1±8.9 mg/kg (45.6±12.1% inhibition of the control RIST) (FIG. 6). The blood pressure was constant throughout the experiment (96.7±4.1 mmHg in control, 93.3±14.3 mmHg after L-NMMA before the RIST and 90.0±9.4 mmHg before the final RIST). After two hours RIST was repeated again and the amount of glucose required to maintain the euglycemia was 76.1±14.8 mg/kg (65.1±13.0% inhibition of the control RIST). Thus, intraportal L-NMMA produces insulin resistance that is maintained for two hours.

RIST after L-NMMA and SIN-1 intravenously or intraportally. Intraportal infusion of L-NMMA (0.73 mg/kg, n=4) significantly reduced the RIST index from 218.4±6.6 mg/kg to 88.4±21.6 mg/kg (59.6±9.7% inhibition of the control RIST). Intravenous administration of SIN-1 (5.0 mg/kg) did not reverse inhibition caused by L-NMMA (59.0±7.2% inhibition ) (FIG. 7). In the second set of animals (n=5), the control RIST index was 236.9±20.0 mg/kg. Intraportal infusion of L-NMMA (0.73 mg/kg) caused significant insulin resistance and reduced the RIST index to 129.7±14.3 mg/kg and caused (54.5±2.0% inhibition)(FIG. 7). Intraportal SIN-1 (5.0 mg/kg) partially reversed the inhibition caused by L-NMMA (24.0±11.6%). Thus, NO production in the liver can partially reverse insulin resistance caused by NOS antagonism.

RIST after L-NMMA and intraportal SIN-1. Intraportal infusion of L-NMMA (0.73 mg/kg, n=5) significantly reduced the RIST index from 221.34±30.9 mg/kg to 99.3±20.9 mg/kg (55.5±7.0% inhibition of the control RIST). Intraportal SIN-1 (10.0 mg/kg) completly reversed the inhibition caused by L-NMMA (0.6±5.8%) (FIG. 8). Thus, higher NO production in the liver can completely reverse insulin resistance caused by NOS antagonism.

RIST after denervation and intraportal SIN-1 (n=6). Surgical denervation of the hepatic anterior plexus significantly reduced the RIST index from 208.3±15.0 mg/kg to 87.7±10.3 mg/kg (56.4±6.7% inhibition of the control RIST). Intraportal SIN-1 (10.0 mg/kg) completly reversed the inhibition caused by denervation (3.8±10.4/%) (FIG. 9). Thus, NO production in the liver can reverse insulin resistance caused by surgical denervation of the liver.

### DISCUSSION

Previous studies (27-29) are consistent with the statement that animals respond to insulin by activation of a hepatic parasympathetic reflex release of a hepatic insulin sensitizing substance (HISS) that sensitizes skeletal muscle to the effects of insulin. Surgical or pharmacological ablation of the hepatic parasympathetic nerves leads to insulin resistance. Intraportal, but not intravenous, Ach is capable of reversing the insulin resistance caused by denervation. The hepatic parasympathetic reflex control of insulin action is mediated through hepatic NO and hepatic NOS antagonism and hepatic denervation produce insulin resistance that is reversible by providing NO to the liver using a NO donor. The parasympathetic reflex release of HISS is concluded to be NO-mediated.

Technical considerations. The rapid insulin sensitivity test (RIST) is a modified euglycemic clamp method (11,30). Insulin (50 mU) is infused over five minutes and the total amount of glucose infused (RIST index) in order to maintain arterial glucose at the baseline level during the 30 minutes of the test is used to express insulin sensitivity in each test. The difference between a control RIST and the RIST index after surgical hepatic denervation or atropine is used to determine the hepatic parasympathetic component of insulin action (27, 29). Three RISTs were performed, as time controls, in the same rat during one experiment with a coefficient of variance of 8.8±1.5%. The basal glucose levels before each RIST were not significantly different. The blood pressure was stable throughout and between each test. The RIST is sensitive and shows inhibition by L-NAME, L-NMMA, atropine and hepatic denervation in anesthetized animals.

It had been shown that L-NAME is both a NOS inhibitor and a muscarinic receptor antagonist (2). Although the mechanism or location of action was not described, it was previously determined that L-NAME produces insulin resistance that does not act through muscarinic antagonism (22), thus indicating that both L-NAME and L-NMMA are suitable tools for the present purpose.

Administration of intraportal L-NAME at 1.0 mg/kg causes significant insulin resistance (22). In the present study those data are confirmed using two additional doses of L-NAME, 2.5 mg/kg and 5.0 mg/kg. Administration of L-NAME intravenously at 2.5 mg/kg and 5.0 mg/kg caused significant and similar degrees of insulin resistance. However the effect of the low dose wore off within one hour whereas the high dose effect lasted for more than two hours (FIG. 1). An equimolar dose of L-NMMA to the dose 1.0 mg/kg of L-NAME had a duration of action of at least two hours (FIG. 6).

Nitric oxide synthase inhibition. Reports from other investigators (1) suggest that inhibition of NOS by L-NMMA causes a reduction in skeletal muscle perfusion and this has been suggested as the mechanism of insulin resistance. In these experiments, intraportal L-NMMA (0.73 mg/kg) did not result in hypertension (arterial pressure of 90±3.8 mmHg in control and 84.3±4.6 mmHg after L-NMMA), however significant insulin resistance occurred (FIG. 6). Oral administration of L-NAME caused hypertension but not insulin resistance (26), showing that insulin resistance is not a result of vascular effects but of a fundamental metabolic disorder. Surgical hepatic denervation significantly reduced insulin sensitivity and subsequent NOS inhibition with L-NMMA did not cause additional insulin resistance (FIG. 4). If the NOS antagonist effect was secondary to peripheral effects it should have been additive to the effects of liver denervation. This observation shows that hepatic parasympathetic interruption by surgery or NOS inhibition in the liver caused insulin resistance by interruption of the same pathway.

To confirm the site of action of L-NAME intraportal infusion of L-NAME dose (1.0 mg/kg) was compared with intravenous infusion of the same dose. The intraportal, but not intravenous, dose caused significant insulin resistance. The observation that L-NAME caused more insulin resistance when administered intraportally (FIG. 3) shows that the site of action of L-NAME is the liver.

Insulin resistance caused by NOS antagonism is not a result of reduction in skeletal muscle perfusion but rather is caused by blockade of the parasympathetic reflex release of a hepatic factor that is released in response to insulin. This putative hepatic insulin sensitizing substance (HISS) amplifies the skeletal muscle response to insulin (28) and hepatic NOS inhibition interrupts this pathway.

Vasodilatory effect of insulin. Insulin-mediated vasodilation increases glucose uptake in skeletal muscles (5,18,24). However, Scherrer et al. (23) showed that L-NMMA, when infused into one arm, reduces forearm blood flow and increases blood pressure, but does not alter the whole-body glucose uptake (24). Natali et al. demonstrated that increasing forearm blood flow with sodium nitroprusside in obese hypertensive patients does not improve insulin sensitivity (15,16). Mijares et al. concluded that after a mixed meal, skeletal muscle blood flow does not increase enough for blood flow to be a major contributor to glucose uptake (13). The effect of insulin on blood flow is controversial. Some investigators report increased blood flow only at high, supraphysiological insulin concentrations (19). Most investigators (1) use the hyperinsulinemic euglycemic clamp technique to measure insulin sensitivity. In this technique, insulin is infused at a constant rate for 2-3 hrs before steady state conditions are achieved. It is possible that infusion of insulin for long periods of time and at high concentrations results in vasodilation and increased blood flow. However the insulin used in these experiments, given over five minutes, is short acting and the RIST is completed by 30 minutes. Baron et al. (1) report that during the hyperinsulinemic euglycemic technique there is a fall in mean arterial pressure caused by the vasodilatory effect of insulin. In these experiments there was no significant change in blood pressure during insulin administration. Furthermore, if NOS antagonism produced insulin resistance secondary to direct blockade of dilatory responses to insulin in skeletal muscle, the intravenous dose should have produced a greater effect than the intraportal dose, the opposite of these findings (FIG. 3). Similarly the ability of intraportal but not intravenous NO donor to reverse L-NMMA-induced insulin resistance indicates that the drugs are acting through the liver. Further, if NOS antagonism produced insulin resistance secondary to blocking vascular responses to insulin in skeletal muscle, the insulin resistance caused by hepatic denervation should have been made worse by the addition of this peripheral effect. Insulin resistance produced by denervation was not affected by addition of a NOS antagonist. Thus, in these testing conditions the data are consistent with insulin resistance following NOS antagonism being secondary to a hepatic, rather than peripheral, effect.

Reversal of insulin resistance. L-arginine did not produce the anticipated reversal of insulin resistance produced by L-NAME, but rather L-arginine, by itself, caused insulin resistance (48.8±8.2%) (FIG. 5). L-NAME not only blocks NOS but also blocks arginine uptake across the hepatocyte plasma membrane (8). L-arginine is metabolized by NOS to NO, and by arginase to urea and L-ornithine (6). Since the liver has a very high arginase activity, most L-arginine administered is converted to L-ornithine by the liver, although L-arginine can reverse the vascular effects of L-NAME in the liver (12). L-arginine also causes release of growth hormone (7,14) and glucagon; both hormones reduce insulin sensitivity. This explains why insulin resistance caused by L-NAME could not be reversed with L-arginine and why L-arginine caused insulin resistance.

Reduction in blood flow to the nerves in diabetes leads to neuropathy (3,4,9,17, 25) and has been shown to result from a decrease in NO production in the vasculature (3, 9). Administration of L-NAME in normal rats decreased nerve blood flow that was reversed by L-arginine (9,17). L-NAME also caused basal vasoconstriction in the intestine that was reversible by L-arginine (12).These observations show that L-arginine is capable of reversing the effect of L-NAME in the vasculature. This shows that acute insulin resistance caused by L-NAME is not secondary to effects on perfusion of hepatic nerves or peripheral blood vessels since it was not reversed with L-arginine.

As an alternative to using L-arginine to reverse the effect of NOS blockade, the NO donor, SIN-1, was used. Administration of intraportal, but not intravenous, SIN-1 (5.0 mg/kg) partially reversed the insulin resistance caused by L-NMMA (FIG. 7). However, administration of a higher dose of SIN-1 (10.0 mg/kg) to the liver completely reversed the insulin resistance caused by L-NMMA (FIG. 8). This indicates that insulin resistance produced after inhibition of NOS in the liver can be reversed by providing NO in the liver. Also, administration of intraportal SIN-1 after denervation of the liver completely restored insulin sensitivity (FIG. 9). Thus, NO production in the liver is confirmed to be essential for insulin sensitivity.

Reversal of denervation-induced insulin resistance is additional evidence that the parasympathetic reflex involves a hormonal pathway. If there was a neural connection between the liver and skeletal muscle that was controlling insulin sensitivity, this connection has been severed in order to produce the insulin resistance. Administration of SIN-1 into the portal vein cannot restore the response by a reflex pathway since the relevant nerves have been cut.

HISS-dependent and -independent effect. The RIST index in control responses and the reduction in control RIST index after atropine or denervation was examined by linear regression as previously reported (29). The rats showing the highest control RIST index had the greatest reduction in response after atropine or denervation, and rats showing the lowest control RIST index had the smallest decrease in control RIST index (FIG. 4b). The decrease in the RIST after denervation or atropine represents the HISS-dependent component of insulin action. This shows a parasympathetic-dependent component (to the right of the x-intercept) and a parasympathetic-independent component (the x-intercept) of insulin action. A similar relationship is observed after L-NAME administration. After L-NAME, the rats showing high control RIST indexes had large decreases in the RIST index, and the rats showing small control RIST indexes had small decreases in the RIST index (FIG. 2a). This shows a hepatic NO-dependent component and a NO-independent component involved in insulin action. The regression analysis is not significantly different in slope or intercept using the combined atropine and denervation data compared to the NOS blockade data. There is a parasympathetic-dependent and -independent and also a NO-dependent and -independent component involved in insulin responsiveness and both components act through the same pathway. This pathway is shown to consist of an insulin-induced hepatic parasympathetic reflex, acting through muscarinic receptors, resulting in production of NO in the liver, leading to release of the putative hormone, HISS, that sensitizes the skeletal muscle to the action of insulin. Interruption of this NO-mediated reflex inhibits HISS release from the liver and insulin resistance follows.

In conclusion, there is a strong relationship between inhibition of NOS in the liver and insulin resistance. Providing NO to the liver reverses this insulin resistance. Therefore, inhibition of the NOS in the liver interrupts the HISS pathway and, because HISS is needed to sensitize the skeletal muscle response to insulin, insulin resistance occurs.

### EXAMPLE 2

An insulin sensitivity test showing amount of glucose needed to be administered after insulin (50 mU/kg i.v.) in order to maintain arterial glucose steady is analyzed. In group 1, a nitric oxide synthase blocker (blocks production of nitric oxide), L-NAME, was given into the portal vein and produced a 54.9±5.2% inhibition of insulin response. Atropine, in a dose known to produce full blockade of the hepatic parasympathetic nerves, was administered intravenously after L-NAME and produced a modest further resistance (67.2±4.9%). In a separate group of rats, the same dose of L-NAME was given intravenously and did not produce significant insulin resistance (19.8±7.5%). The blockade of muscarinic receptors with atropine produced normal insulin resistance (56.0±8.9%) expected from parasympathetic interruption. The data show conclusively that insulin resistance produced by blockade of NO synthase did so by acting on the liver rather than other tissues.

### EXAMPLE 3

Insulin resistance (45.0±3.0% of normal response) is produced by the blockade of nitric oxide synthase (eliminates production of nitric oxide) which is not reversed by administration of a nitric oxide donor intravenously but is fully reversed by administration of the same dose directly to the liver via the portal vein. This response conclusively shows that the liver is the site of nitric oxide regulation of insulin sensitivity.

### EXAMPLE 4

The hypothesis explaining hepatic parasympathetic reflex release of hepatic insulin sensitizing substance (HISS) from the liver in response to insulin, is that HISS is mediated by hepatic cholinergic receptors and nitric oxide (NO) release. In absence of either nerve function or NO releasem, severe insulin resistance occurs. Depending upon the pathology, the resistance can be restored to normal by administration of a cholinergic agonist or a source of nitric oxide.

Throughout this application, various publications, including United States patents, are referenced by citation or number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### REFERENCES

1-Baron, A. D., J. S. Zhu, S. Marshall, O. Irsula, G. Brechtel, and C. Keech. Insulin resistance after hypertension induced by the nitric oxide synthesis inhibitor L-NMMA in rats. Am. J. Physiol. 269:E709-E715, 1995.

2-Buxton, I. L. O., D. J. Cheek, D. Eckman, D. P. Wesffall, K. M. Sanders, and K. D. Keef. N-nitro L-arginine methyl ester and other alkyl esters of arginine are muscarinic receptor antagonists. Circ. research. 72: 387-395, 1993.

3-Cameron, N. E. and M. A. Cotter. Effects of chronic treatment with a nitric oxide donor on nerve conduction abnormalities and endoneurial blood flow in streptozotocin-diabetes rats. Eur. J. Clin. Invest. 25: 19-24, 1995.

4- Cameron, N. E., M. A. Cotter, and T. C. Hohman. Interactions between essential fatty acids, prostanoid, polyol pathways and nitric oxide mechanisms in the neurovascular deficit of diabetic rats. Diabetologia. 39: 172-182, 1996.

5-Chen Y. L. and E. J. Messina. Dilation of isolated skeletal muscle arterioles by insulin is endothelium dependent and nitric oxide mediated. Am. J. Physiol. 270: H2120-H2124, 1994.

6-Cook, H. T., A. Jansen, S. Lewis, P. Largen, M. O'Donnell, D. Reaveley, and V. Cattell. Arginine metabolism in experimental glomerulonephritis: interaction between nitric oxide synthase and arginase. Am. J. Physiol. 267: F646-F653, 1994.

7- Cyber, C. Can arginine and ornithine support gut function? Gut (supplement). 1: S42-S45,1994.

8-Inoue, Y., B. P. Bode, D. J. Beck. A. P. Li, K. I. Bland, and W. W. Souba. Arginine transport in human liver. Ann. Surgery. 218(3): 350-363,1993.

9-Kihara, M. and P. A. Low. Impaired vasoreactivity to nitric oxide in experimental diabetic neuropathy, Exp. Neurol. 132:180-185, 1995.

10-Lautt, W. W. Hepatic parasympathetic neuropathy as cause of maturity-onset diabetes? Gen. Pharmacol. 11:343-345, 1980.

11-Lautt, W. W., X. Wang, P. Sadri, D. J. Legare, and M. P. Macedo. Rapid insulin sensitivity test (RIST). Can. J. Physiol. Pharmacol. 76: 1080-1086, 1998.

12-Macedo, M. P. and W. W. Lautt. Autoregulatory capacity in the superior mesenteric artery is attenuated by nitric oxide. AM. J. Physiol. 271: G400-G404, 1996.

13-Mijare, A. H. and M. D. Jensen. Contribution of blood flow to leg glucose uptake during a mixed meal. Diabetes. 44: 1165-1169,1995.

14-Nakaki, T. and R. Kato. Beneficial circulatory effects of L-arginine. Jpn. J. Pharmacol. 66:167-171,1994.

15- Natali, A. Skeletal muscle blood flow and insulin action. Nutr. Metab. Cardio. Dis. 7: 105-109, 1997.

16-Natali, A., A. Q. Galvan, N. Pecorri, G. Sanna, E. Toschi, and E. Ferrannini. Vasodilation with sodium nitroprusside does not improve insulin action in essential hypertension. Hypertension. 31: 632-636, 1998.

17- Omawari, N., M. Dewhurst, P. Vo, S. Mahmood, E. Stevens, and D. R. Tomlinson. Deficient nitric oxide responsible for reduced nerve blood flow in diabetes rats: effects of L-Name, L-arginine, sodium nitroprusside, and evening primrose oil. Br. J. Pharmacol. 118:186-190, 1996.

18-Pitre, M., A. Nadeau, and H. Bachelard. Insulin sensitivity and hemodynamic responses to insulin in wistar-kyoto and spontaneously hypertensive rats. Am J. Physiol. 271:E658-E668, 1996.

19-Porter, J. P., I. G. Joshua, D. Kabith, and H. S. Bokil. Vasodilator effect of insulin on the microcirculation of the rat cremaster muscle. Life Sciences. 61(7): 673-687,1997.

20- Raitakari, M., P. Nuutila, U. Ruotsalainen, H. Lain, M. Teras, H. Iida, S. Makimattila, T. Utriainen, V. Oikonen, H. Sipila, M. Haaparanta, O. Solin, U. Wwegelius, J. Knuuti, and H. Yki-Jarvinen. Evidence for dissociation of insulin stimulation of blood flow and glucose uptake in human skeletal muscle. Diabetes. 45:1471-1477,1996.

21- R e, D. D., R. M. J. Palmer, R. Schulz, H. F. Hods n, and S. Moncada. Characterization of three inhibitors of endothelial nitric oxide synthase in vitro and in vivo. Br. J. Pharmacol. 101:746-752, 1990.

22- Sadri, P. and W. W. Lautt. Blockade of nitric oxide production in the liver causes insulin resistance. Proc. West. Pharmacol. Soc. 1998.

23- Scherrer, U., D. Randin, P. Vollenweider, L. Vollenweider, and P. Nicod. Nitric oxide release accounts for insulin's vascular effects in humans. J. Clin. Invest. 94: 2511-2515, 1994.

24- Steinberg, H. O., G. Brechtel, A. Johnson, N. Fineberg, and A. D. Baron. Insulin-mediated skeletal muscle vasodilation is nitric oxide dependent. J. Clin. Invest. 94:1172-1179,1994.

25-Stevens, M. J. Nitric oxide as a potential bridge between the metabolic and vascular hypotheses of diabetic neuropathy. Diabetic Med. 12: 292-295, 1995.

26-Swislocki, A., T. Eason, and G. A. Kayen. Oral administration of nitric oxide biosynthesis inhibitor, N-Nitro-L-Arginine Methyl Ester (L-NAME), causes hypertension, but not glucose intolerance or insulin resistance, in rats. AJH 8:1009-1014, 1995.

27-Xie, H. and W. W. Lautt. Induction of insulin resistance by cholinergic blockade with atropine in the cat. J. Autono. Pharmacol. 15: 361-369, 1995.

28- Xie, H. and W. W. Lautt. Insulin resistance of skeletal muscle produced by hepatic parasympathetic interruption. Am. J. Physiol. 270: E858-E863, 1996.

29- Xie, H. and W. W. Lautt. Insulin resistance caused by hepatic cholinergic interuption and reversed by acetylcholine administration. Am. J. Physiol. 271: E587-E592,1996.

30- Xie, H., L. Zhu, Y. L. Zhang, D. J. Legare, and W. W. Lautt. Insulin sensitivity tested with modified euglycemic technique in cats and rats. J. Pharmacol. Toxicol. Methods. 35:77-82, 1996.

1. A method of increasing insulin sensitivity by:
administering an effective amount of a compound which stimulates nitric oxide production in the liver.

2. The method according to claim 1, wherein said administering step further includes orally administering the compound.

3. The method according to claim 1, wherein said administering step further includes injecting the compound.

4. The method according to claim 1, wherein said administering step further includes delivering the compound through a pump system directly into the portal vein.

5. A pharmaceutical composition comprising an effective amount of a compound which stimulates nitric oxide production in the liver and a pharmaceutically acceptable carrier.

6. The pharmaceutical composition according to claim 5, wherein said compound is a nitric oxide agonist.

7. The pharmaceutical composition according to claim 5, wherein said compound is a nitric oxide donor.

8. The us of a compound which stimulates nitric oxide production in the liver and a pharmaceutically acceptable carrier.
